(19) United States
(12) Patent Application Publication
(10) Pub. No.: US 2023/0097795 A1
(43) Pub. Date: Mar. 30, 2023

(54) PANT-TYPE WEARABLE ARTICLE

(71) Applicant: The Procter & Gamble Company, Cincinnati, OH (US)

(72) Inventors: Chunmin CHENG, Beijing (CN); Koichi MORIMOTO, Beijing (CN); Mark Thomas STILLWAGON, Hamilton, OH (US); Qilei YIN, Beijing (CN); Claus Peter STOELZEL, Bad Soden (DE)

(21) Appl. No.: 18/072,786

(22) Filed: Dec. 1, 2022

Related U.S. Application Data
(63) Continuation of application No. PCT/CN2021/108419, filed on Jul. 26, 2021.

(30) Foreign Application Priority Data
Jul. 27, 2020 (CN) PCT/CN2020/104810

(51) Int. Cl. A61F 13/496; A61F 13/49

(52) U.S. Cl. CPC A61F 13/496; A61F 13/49009

(57) ABSTRACT

Disclosed is a wearable article comprising a front elastic belt region, a back elastic belt region, each of the front and back elastic belt region comprises a laminate comprising an inner sheet, an outer sheet, and a plurality of elastic members running in the transverse direction, the longitudinal dimension between adjacent elastic members forming an elastic spacing; wherein the laminate is bonded by heat at the side edges to form side seams, the side seams existing no distant than about 20 mm from the side edges, and the remainder of the laminate is substantially free of heat bonds, wherein the article has a Belt Elastic Bonding Percentage of less than about 25%, and an Individual Elastic Bonding Percentage of from about 10% to about 75%, according to the measurements herein.

### CROSS REFERENCE TO RELATED APPLICATIONS

This application is a continuation of, and claims priority under 35 U.S.C. § 120 to, Patent Application No. PCT/CN2021/108419, filed on Jul. 26, 2021, which claims priority to Patent Application No. PCT/CN2020/104810, filed on Jul. 27, 2020, the entire disclosures of which are hereby incorporated by reference.

### FIELD OF THE INVENTION

The present invention relates to pant-type wearable articles having an elastic belt with, improved comfort, improved softness, and improved function intuitive appearance, while providing good fit.

### BACKGROUND OF THE INVENTION

Infants and other incontinent individuals wear absorbent articles such as diapers to receive and contain urine and other body exudates. Pull-on absorbent articles, or pant-type absorbent articles, are those which are donned by inserting the wearer's legs into the leg openings and sliding the article up into position about the lower torso. Pant-type absorbent articles have become popular for use on children who are able to walk and often who are toilet training, as well as for younger children who become more active in movement such that application of taped-type absorbent articles tends to be more difficult, and also for younger babies requiring a soft fit around the waist opening and leg openings.

Pant-type articles may take various structures wherein the circumference of the waist opening and vicinity thereof is made elastic enough to facilitate the wearer or the caregiver to expand the article and insert the wearer's legs into the leg openings for wearing the article. The region of the waist circumference and vicinity thereof is often referred to as the elastic belt. One type of structure for the pant-type article is the belt-type pant having a central chassis to cover the crotch region of the wearer and a separate elastic belt defining the waist opening and leg opening, such as described in PCT Publication WO 2006/17718A. Another type of structure for the pant-type article is the uni-body pant configured such that the outer cover of the article completely covers the entirety of the garment-facing surface of the article, wherein the portion configured to stretch about the torso is considered the elastic belt region.

Whatever the structure of the pant-type article may be, pant-type articles provide only a very small range of size adjustment or body configuration adjustment based on the structural limitations of the article. As such, pant-type articles are typically so configured to accommodate size and configuration ranges by providing the elastic belt region very stretchable and comfortable to wear, yet with reliable fit such that sufficient protection against sagging and leakage may be provided. Further, the elastic belt region may be the portion which is most touched and observed by the wearer or the caregiver upon use, and thus its properties most associated with the function and quality of the article. An appearance that intuitively connotes the function of the article, or the function of a particular part of the article is advantageous.

Based on the foregoing, there is a need for a wearable article providing improved softness, improved stretchability for ease of application, improved fit for preventing sagging, improved comfort and improved breathability for skin health. There is also a need for a wearable article having improved function intuitive appearance. There is also a need for providing such a wearable article which can be economically made.

### SUMMARY OF THE INVENTION

The present invention is directed to a wearable article continuous in a longitudinal direction and a transverse direction comprising a front elastic belt region, a back elastic belt region, a crotch region, a waist opening, and a pair of leg openings; the crotch region extending longitudinally between the front elastic belt region and the back elastic belt region;

wherein each of the front and back elastic belt region comprises a laminate comprising an inner sheet, an outer sheet, and a plurality of elastic members running in the transverse direction, the longitudinal dimension between adjacent elastic members forming an elastic spacing;

wherein the laminate further comprising an elastic bonding which continuously bonds the elastic members for at least about 10 mm in the direction of stretch in a region adjacent the side edges of the front and back elastic belt regions, and a plurality of discrete bond units disposed between the elastic bondings in the transverse direction, each discrete bond unit applied to at least one of the inner sheet and the outer sheet and having a longitudinal dimension of from about 0.5 mm to about 2.0 mm, and a transverse dimension of from about 0.5 mm to about 2.0 mm, wherein between any two discrete bond units, the discrete bond units have a longitudinal spacing of at least about 0.2 mm with each other, and a transverse spacing of at least about 0.2 mm with each other, wherein the plurality of discrete bond units form a plurality of longitudinal columns, wherein at least about 30%, preferably at least about 50%, of the longitudinal columns have a collective spacing of discrete bond units that are not constant, and the collective spacing of discrete bond units of any adjacent longitudinal columns are different from each other;

wherein there is at least one discrete bond unit disposed in each elastic spacing;

wherein the laminate is bonded by heat at the side edges to form side seams, the side seams existing no distant than about 20 mm from the side edges, and the remainder of the laminate is substantially free of heat bonds,

wherein the article has a Belt Elastic Bonding Percentage of less than about 25%, and an Individual Elastic Bonding Percentage of from about 10% to about 75%, preferably from about 20% to about 60%, according to the measurements herein.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings and which like designations are used to designate substantially identical elements, and in which:

FIG. 1A is a perspective view of one embodiment of a wearable article of the present invention.

FIG. 1B is a schematic view of one embodiment of a wearable article of the present invention in a contracted state showing the front side of the article.

FIG. 2 is a schematic plan view of one embodiment of a wearable article of the present invention with the seams unjoined and in a flat uncontracted condition showing the garment facing surface.

FIG. 3A is a schematic plan view of one embodiment of FIG. 2 showing the elastic member positioning, elastic adhesive bondings and area where plurality of discrete bond units are disposed.

FIG. 3B is an expanded schematic plan view of FIG. 3A showing one embodiment of a pattern of discrete bond units.

FIG. 3C is an expanded schematic plan view of FIG. 3A showing another embodiment of a pattern of discrete bond units.

FIG. 3D is an expanded schematic plan view of FIG. 3A showing another embodiment of a pattern of discrete bond units.

FIG. 3E is an expanded schematic plan view of FIG. 3A showing another embodiment of a pattern of discrete bond units.

FIG. 3F is an expanded schematic plan view of FIG. 3A showing another embodiment of a pattern of discrete bond units.

FIG. 4 is a schematic view of an example of a hanger-type sample holding fixture according to the “Whole Article Force Measurement”.

### DEFINITIONS

As used herein, the following terms shall have the meaning specified thereafter:

“Wearable article” refers to articles of wear which may be in the form of pants, taped diapers, incontinent briefs, feminine hygiene garments, and the like. The “wearable article” may be so configured to also absorb and contain various exudates such as urine, feces, and menses discharged from the body. The “wearable article” may serve as an outer cover adaptable to be joined with a separable disposable absorbent insert for providing absorbent and containment function, such as those disclosed in PCT publication WO 2011/087503A.

“Pant” refers to disposable absorbent articles having a pre-formed waist and leg openings. A pant may be donned by inserting a wearer's legs into the leg openings and sliding the pant into position about the wearer's lower torso. Pants are also commonly referred to as “closed diapers”, “prefastened diapers”, “pull-on diapers”, “training pants” and “diaper-pants”.

“Longitudinal” refers to a direction running substantially perpendicular from a waist edge to an opposing waist edge of the article and generally parallel to the maximum linear dimension of the article.

“Transverse” refers to a direction perpendicular to the longitudinal direction.

“Proximal” and “distal” refer respectively to the position closer or farther relative to the longitudinal center of the article.

“Body-facing” and “garment-facing” refer respectively to the relative location of an element or a surface of an element or group of elements. “Body-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

“Disposed” refers to an element being located in a particular place or position.

“Joined” refers to configurations whereby an element is directly secured to another element by affixing the element directly to the other element and to configurations whereby an element is indirectly secured to another element by affixing the element to intermediate member(s) which in turn are affixed to the other element.

“Film” refers to a sheet-like material wherein the length and width of the material far exceed the thickness of the material. Typically, films have a thickness of about 0.5 mm or less.

“Water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure (aside from natural forces such as gravity). A layer or a layered structure that is water-impermeable according to this definition may be permeable to water vapor, i.e., may be “vapor-permeable”.

“Extendibility” and “extensible” mean that the width or length of the component in a relaxed state can be extended or increased.

“Elasticated” and “elasticized” mean that a component comprises at least a portion made of elastic material.

“Elongatable material”, “extensible material”, or “stretchable material” are used interchangeably and refer to a material that, upon application of a biasing force, can stretch to an elongated length of at least about 110% of its relaxed, original length (i.e. can stretch to 10 percent more than its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongatable material will be considered to be “elastic” or “elastomeric.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

“Dimension”, “Length”, “Width”, “Pitch”, “Diameter”, “Aspect Ratio”, “Angle”, and “Area” of the article are all measured in a state wherein the article is extended to the Full Stretch Circumference W1 according to the “Whole Article Force Measurement” herein, and utilizing a ruler or a loupe, unless specified otherwise.

“Artwork” refers to a visual presentation to the naked eye, which is provided by printing or otherwise, and having a color. Printing includes various methods and apparatus well known to those skilled in the art such as lithographic, screen printing, flexographic, and gravure ink jet printing techniques.

“Color” or “Colored” as referred to herein includes any primary color except color white, i.e., black, red, blue, violet, orange, yellow, green, and indigo as well as any declination thereof or mixture thereof. The color white is defined as those colors having a L* value of at least 94, an a* value equal to 0±2, and a b* value equal to 0±2 according to the CIE L* a* b* color system.

### DETAILED DESCRIPTION OF THE INVENTION

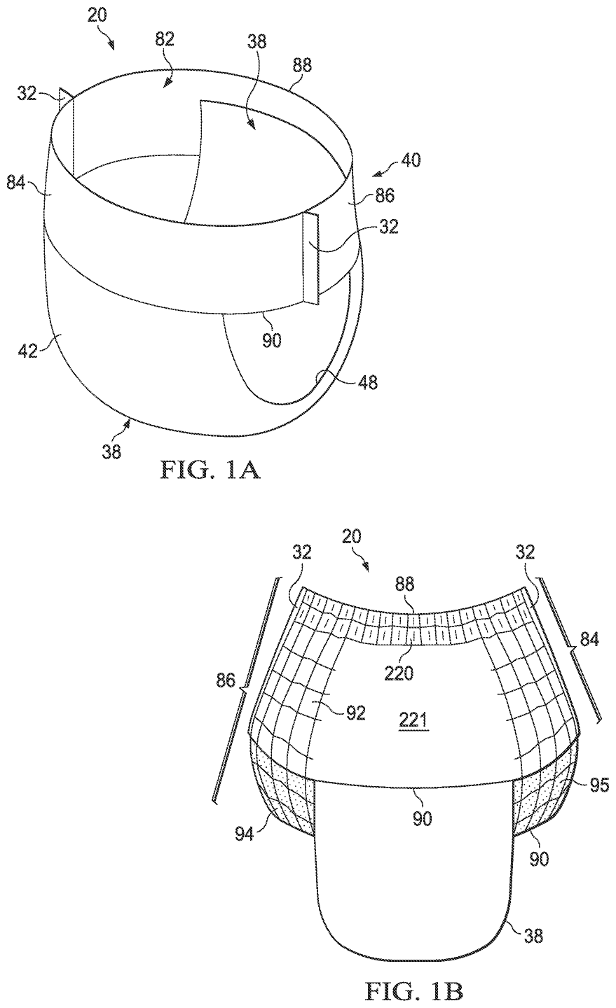

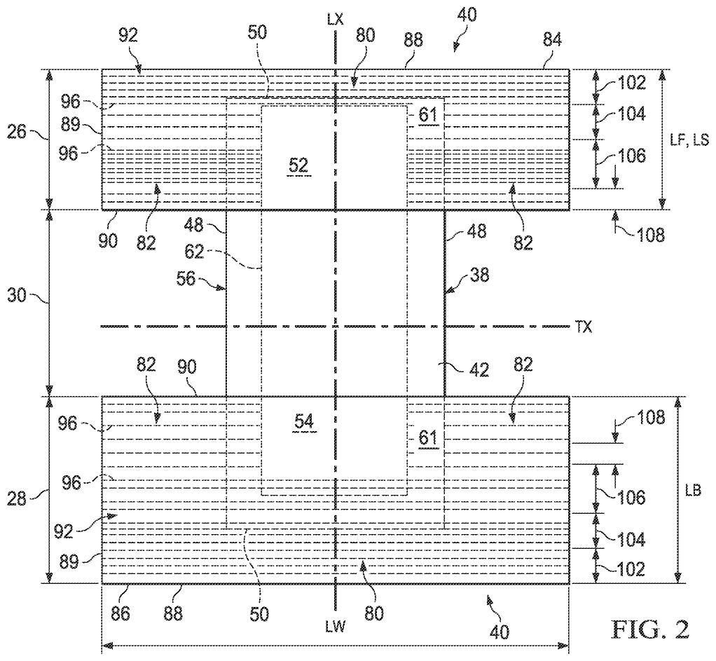

FIG. 1A is a perspective view of a wearable article (20) of the present invention, FIG. 1B is a schematic view of a wearable article of the present invention in a contracted state showing the front side, and FIG. 2 is a schematic plan view of a wearable article with the seams unjoined and in its flat uncontracted condition showing the garment-facing surface. The wearable article (20) has a longitudinal centerline LX which also serves as the longitudinal axis, and a transverse centerline TX which also serves as the transverse axis. The wearable article (20) has a body facing surface, a garment facing surface, a front elastic belt region (84), a back elastic belt region (86), a crotch region (30), and side seams (32) which join the front elastic belt region (84) and the back elastic belt region (86), to form two leg openings and a waist opening.

The wearable article (20) may be a belt-type pant as in FIGS. 1A, 1B, and 2 comprising a central chassis 38 to cover the crotch region (30) of the wearer, a front elastic belt (84) and a back elastic belt (86) (hereinafter may be referred to as “front and back elastic belts”), the front and back elastic belts (84, 86) forming a discrete ring-like elastic belt (40) extending transversely defining the waist opening. For the belt-type pant, the discrete ring-like elastic belt (40) may also be referred to as the elastic belt (40). For the belt-type pant as in FIGS. 1A, 1B, and 2, the front and back elastic belts (84, 86) and the central chassis (38) jointly define the leg openings. For the belt-type pant, the front elastic belt (84) is the front region (26), and the back elastic belt (86) is the back region (28), and the remainder is the crotch region (30). While not shown, the wearable article (20) may be a uni-body type pant configured such that the outer cover of the central chassis (38) and the elastic belt (40) are common. For the uni-body type pant, the portion extending in the transverse direction between the side seams (32), respectively, are considered the front region (26) and the back region (28), and the remainder is the crotch region (30). For the uni-body type pant, the front region (26) is considered the front elastic belt region (84), and the back region (28) is considered the back elastic belt region (86).

The central chassis (38) may comprise a topsheet, a backsheet and an absorbent core (62) disposed between the topsheet and the backsheet, and further an outer cover layer (42) for covering the garment-facing side of the backsheet. The topsheet may be a water permeable substrate. The backsheet may be a water impermeable film. The outer cover layer (42) may be a nonwoven sheet. The central chassis (38) may contain an absorbent core (62) for absorbing and containing body exudates disposed on the central chassis (38), and an absorbent material non-existing region (61) surrounding the periphery of the absorbent core (62). The absorbent material non-existing region (61) may be made of the topsheet and/or the backsheet and/or the outer cover layer (42) and/or other parts configuring the central chassis (38). In the embodiment shown in FIG. 2, the central chassis (38) has a generally rectangular shape, left and right longitudinally extending side edges (48) and front and back transversely extending end edges (50). The absorbent core (62) may exist through the entire longitudinal dimension of the crotch region and extending at least partly in the front region (26); or at least partly in both the front and back regions (26, 28). The central chassis (38) may have a front waist panel (52) positioned in the front region (26) of the absorbent article (20), a back waist panel (54) positioned in the back region (28), and a crotch panel (56) between the front and back waist panels (52, 54) in the crotch region (30). The center of the front elastic belt (84) is joined to a front waist panel (52) of the central chassis (38), the center of the back elastic belt (86) is joined to a back waist panel (54) of the central chassis (38), the front and back elastic belts (84, 86) each having a left side panel and a right side panel (82) where the central chassis (38) does not overlap. The central chassis has a crotch panel (56) positioned between the front waist panel (52) and the back waist panel (54).

The absorbent core (62) may include an absorbent layer and an acquisition layer. The absorbent layer is the region wherein absorbent materials having a high retention capacity, such as superabsorbent polymers, are present. The absorbent layer may be substantially cellulose free. Superabsorbent polymers of the absorbent layer may be disposed between first and second layers of material immobilized by a fibrous layer of thermoplastic adhesive material. The first and second layers of materials may be nonwoven fibrous webs including synthetic fibers, such as mono-constituent fibers of PE, PET and PP, multiconstituent fibers such as side by side, core/sheath or island in the sea type fibers. Such synthetic fibers may be formed via a spunbonding process or a meltblowing process. The acquisition layer facilitates the acquisition and the distribution of body exudates and may be placed between the topsheet and the absorbent layer. The acquisition layer may include cellulosic fibers.

The absorbent layers may be disposed in plurality in the absorbent core (62). Some portions of the absorbent layers may be configured to have substantially no absorbent material to form a channel or a plurality of channels. Channels may be useful for allowing the absorbent core (62) to bend upon swelling with fluids, such that the absorbent article conforms to the wearer's body after swelling and prevent sagging of the article. The channels may also be formed in the acquisition layer, and may be configured to at least partly match the channels of the absorbent layer in the thickness direction.

The elastic belt (40) of the article of the present invention acts to dynamically create fitment forces and to distribute the forces dynamically generated during wear. The front and back elastic belts (84, 86) may be joined with each other only at the side edges (89) to form side seams (32), a waist opening and two leg openings. Each leg opening may be provided with elasticity around the perimeter of the leg opening. The elasticity around the leg opening may be provided by the combination of elasticity from the front belt (84), the back belt (86), and the central chassis (38).

The longitudinal length of the backsheet and the outer cover layer (42) may be the same, or may be varied. For example, the outer cover layer (42) may have a shorter length compared to that of the backsheet, such that the outer cover layer (42) is devoid where the central chassis (38) overlaps the elastic belt (40). By such configuration, the elastic belt may have better breathability. Further, such configuration may provide cost saving. The transverse width of the backsheet and the outer cover layer (42) may be the same, or may be varied. For example, the backsheet may have a shorter transverse width compared to that of the outer cover layer (42). By such configuration, the longitudinal side edges (48) of the crotch panel (56), which make part of the leg openings, may have better breathability. Further, such configuration may provide cost saving.

The front elastic belt (84) and back elastic belt (86) are configured to impart elasticity to the belt (40). Referring to FIGS. 1B and 2, the front belt (84) and the back belt (86) may each comprise a laminate, the laminate comprising a plurality of elastic members (96) running in the transverse direction, an inner sheet (94), an outer sheet (92), and an outer sheet fold over (not shown) wherein the outer sheet fold over is an extension of the outer sheet material formed by folding the outer sheet material at the distal edge (88) of the front and back belts; wherein the belt elastic members (96) are sandwiched between two of these sheets. The longitudinal dimension between adjacent elastic members (96) form an elastic spacing. The front elastic belt (84) and the back elastic belt (86) may each be made only by elastic members (96), the inner sheet (94), the outer sheet (92), and the outer sheet fold over. The belt elastic members (96) may extend in the transverse direction to provide a ring like elastic belt (40) when the front elastic belt (84) and the back elastic belt (86) are joined. At least some of the elastic members (96) extend in the transverse direction substantially parallel to each other. All of the elastic members (96) may extend in the transverse direction substantially parallel to each other. Such an article may be economically made. The front and back elastic belt (84, 86) each may have transversely continuous proximal and distal edges, the proximal edge (90) being located closer than the distal edge (88) relative to the longitudinal center of the article. At least 10%, or at least from about 15% to not more than about 70%, of the front and back elastic belts from the waist opening in the longitudinal direction may be a laminate in active elasticity along the entire transverse dimension LW of the front and back elastic belts (84, 86). Referring to FIGS. 1B and 2, the front and back elastic belts (84, 86) may be treated such that certain regions are removed of its elastic activity to form a non-elastic region (221). For each front and back elastic belt (84, 86), the region overlapping the front and/or back waist panel (52, 54) of the central chassis (38) may be removed of its elastic activity and defining the non-elastic region (221).

The elastic belt region (40) may be closely associated with the function and quality of the article. Thus, materials for forming the elastic belt region (40), as well as the gathering profile of the elastic belt region, are carefully selected by the manufacturer to provide the desired tactile and visible senses. Tactile sense such as flexibility and cushiony touch may enhance perception of high quality. The appearance of gathers may intuitively connote the function of the article, or the function of the elastic belt region (40). For example, relatively big uniform gathers may connote a fluffy and soft feel. For example, a bubble kind of texture may connote a soft and cushiony feel. Further, other functions provided by the laminate such as stretchability for ease of application, comfort and softness, as well as breathability, may enhance the perception provided by the gather appearance. Gathers intentionally provided to have a certain appearance may intuitively communicate the functional benefits described above, and provide the favorable entire usage experience of the article by the user. The user may be the wearer or the caregiver.

The laminate having improved function intuitive appearance of the present invention may be made by selecting the material for making the laminate, and by providing the plurality of discrete bond units (234) in a certain arrangement, which is explained in further detail below. The material for providing the laminate may be selected from a lofty nonwoven material of relatively high basis weight for either the outer sheet (92) or the inner sheet (94), and provide the other of the outer sheet (92) and inner sheet (94) to have a difference in basis weight. Further, the laminate may be made by bonding the elastic members (96) in an appropriate denier, longitudinal pitch, and force; to one or both of the inner sheet (94) and the outer sheet (92).

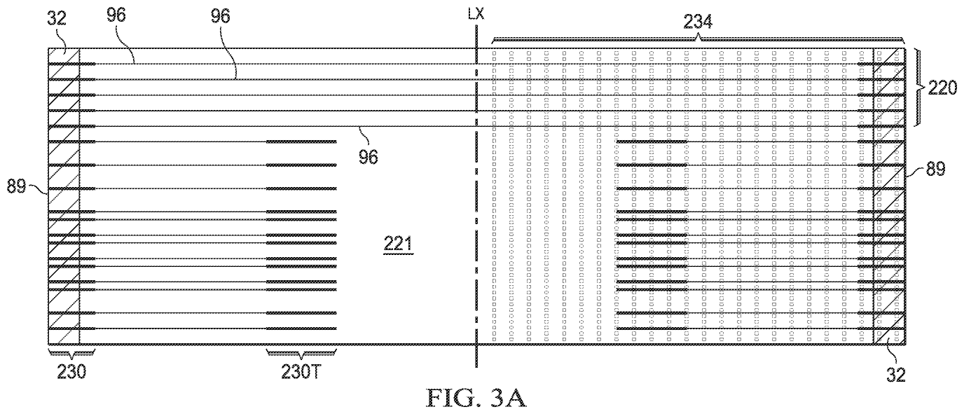

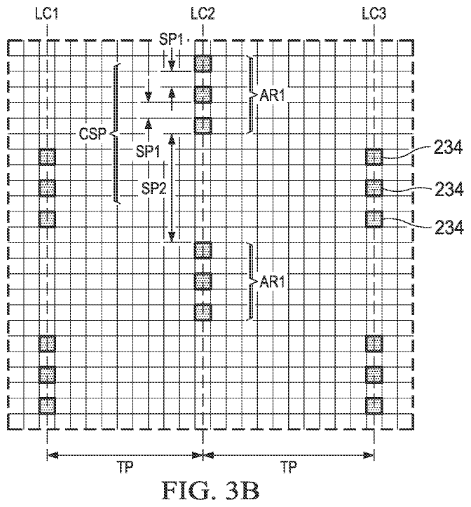

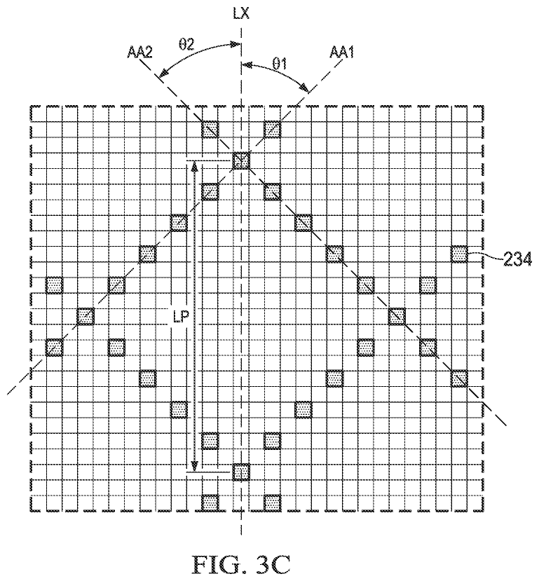

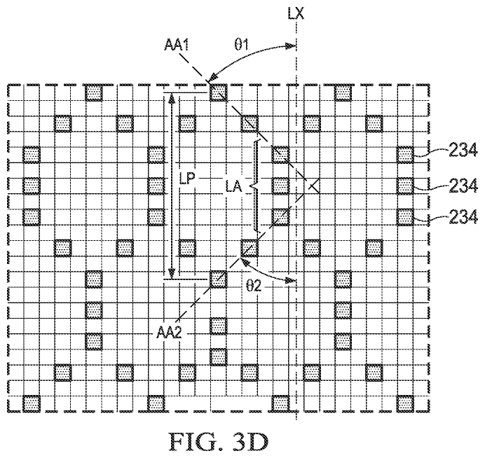

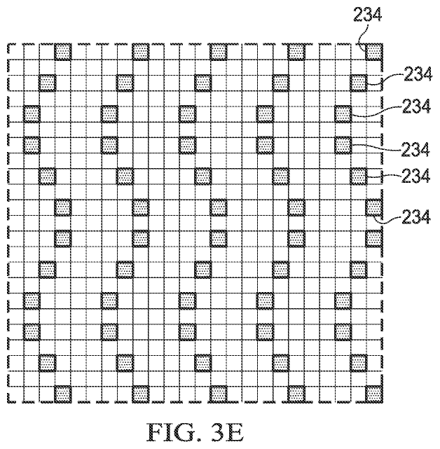

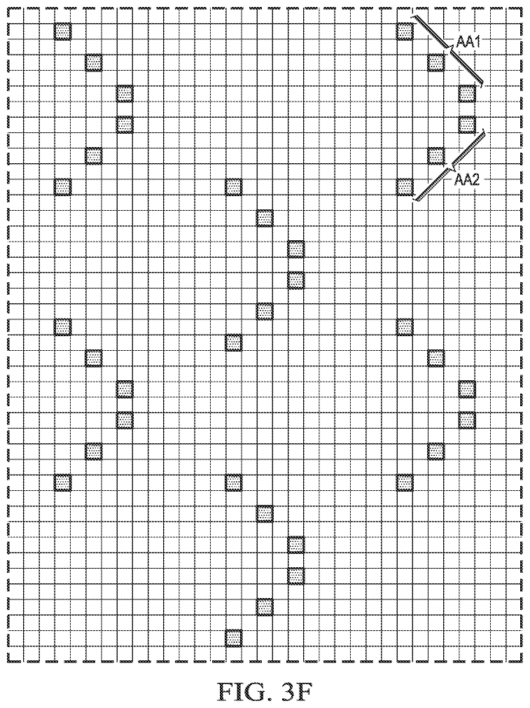

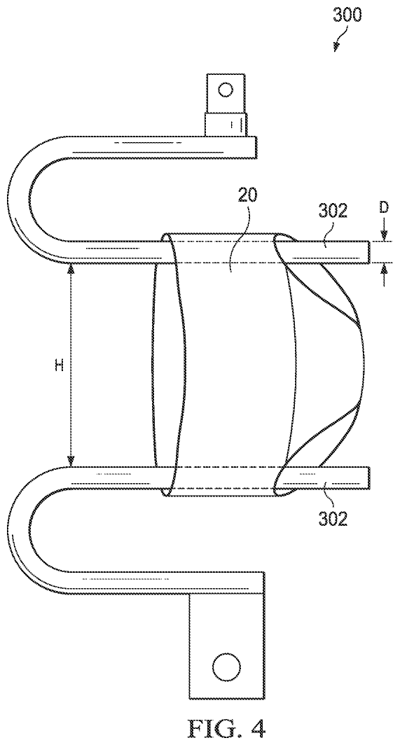

Referring to FIGS. 3A, the laminate may be made by bonding the elastic members (96) to at least one of the inner sheet (94) and the outer sheet (92), via a combination of an elastic bonding (230) and plurality of discrete bond units (234). In FIG. 3A, the front elastic belt (84) is shown with the elastic members (96) and elastic bonding (230) expressed in solid lines. In FIG. 3A, the plurality of discrete bond units (234) is only expressed in the right side of the front elastic belt (84), and the side seams (32) are shown in an unjoined state.

What is meant by elastic bonding (230) herein is an adhesive bonding that bonds the elastic member (96) along the side edges (89) of the front and back elastic belts (84, 86). The elastic bonding (230) may be continuously applied to each elastic member (96) for a length of at least about 10 mm, or from about 10 mm to about 60 mm in the direction of stretch adjacent the side edges (89) of the front and back elastic belts (84, 86), including the length planned for side seaming. The elastic bonding (230) is to provide relatively strong bonding for the elastic member (96) and thus securely anchor the elastic member (96) within the laminate. The anchoring may be assisted by the side seaming A certain percentage, or a greater percentage, of the dimension of the elastic bonding (230) along the side edges (89) may be seamed. The elastic bonding (230) may also be utilized for an effective process of deactivating a limited transverse dimension of the elastic member (96). Referring to FIGS. 2 and 3A, the elastic member (96) may be deactivated in portions overlapping the absorbent core (62). In addition to the side edge regions, the elastic bonding (230T) may be provided on both sides of the certain transverse dimension of the elastic member (96) which is planned to be deactivated, wherein the portion of the elastic member between the elastic bondings (230T) are severed and deactivated. The deactivated portions of the elastic member is not shown in the Figures. Such deactivation may be referred to herein as tummy cut, and the deactivated region may match the non-elastic region (221).

What is meant by plurality of discrete bond units (234) herein is an adhesive bonding applied to at least one of the inner sheet (94) and the outer sheet (92) for intermittently bonding the inner sheet (94) and the outer sheet (92). Each discrete bond unit has a longitudinal dimension of from about 0.5 mm to about 2.0 mm, a transverse dimension of from about 0.5 mm to about 2.0 mm, wherein between any two discrete bond units, the discrete bond units have a longitudinal spacing of at least about 0.2 mm with each other, and a transverse spacing of at least about 0.2 mm with each other. All of the discrete bond units may be provided in the same longitudinal dimension and the same transverse dimension, respectively. Discrete bond units having different longitudinal and/or transverse dimensions may be used. The shape of the bond may be rectangular, circular, or oval.

The plurality of discrete bond units (234) are disposed such that there is at least one discrete bond unit disposed in each elastic spacing. By providing at least one discrete bond unit in each elastic spacing, the elastic members (96) are prevented from contacting each other. In that the elastic bonding (230) provides secure bonding of the elastic member (96) along the side seams (32), as well as the outer periphery of the non-elastic region (221), so long as there is at least one discrete bond unit (234) disposed in each elastic spacing, this prevents the elastic member (96) from moving away from its intended position. The plurality of discrete bond units (234) may also bond the elastic member (96) to at least one of the inner sheet (94) and the outer sheet (92). For an entire front elastic belt (84) or an entire back elastic belt (86), there may be no elastic member (96) bonded to the inner sheet (94) or the outer sheet (92) by a discrete bond unit (234). For an entire front elastic belt (84) or an entire back elastic belt (86), at least one to about 80% of the elastic members (96) may be bonded to the inner sheet (94) or the outer sheet (92) by a discrete bond unit (234). The plurality of discrete bond units (234) may only be provided to the outer sheet (92). The plurality of discrete bond units (234) may only be provided to the inner sheet (94). Referring to FIGS. 3A, the plurality of discrete bond units (234) may be provided for the entire area of the laminate. By providing the plurality of discrete bond units (234) for the entire area of the laminate, the plurality of discrete bond units (234) may serve as a bonding for the inner and outer sheets (92, 94) in regions where the elastic members (96) are severed. The plurality of discrete bond units (234) may be provided in regions adjacent the side edges (89) and thus overlapping the regions where the elastic bondings (230) are provided. Alternatively, the plurality of discrete bond units (234) may be provided only in regions where the elastic bondings (230) are not provided. The plurality of discrete bond units (234) may be provided at least in regions where the elastic member (96) is in active elasticity, wherein the elastic bondings (230) are devoid.

FIGS. 3B-3F describe the planning of discrete bond units (234) for providing a pattern of discrete bond units, as discussed in further detail below. The area showing the pattern for each of FIGS. 3B-3F may vary, namely some Figures may show a larger area than others.

Referring to FIG. 3B, the plurality of discrete bond units (234) form a longitudinal column (LC), which is a group of discrete bond units which fall on a same imaginary line extending parallel to the longitudinal axis. When observing a specific longitudinal column (LC1), there are multiple spacings between the plurality of discrete bond units (234). Such collection of spacings within a specific longitudinal column (LC1) may be constant or may not be constant. As a result of the unique spacing collection within a specific longitudinal column, such specific longitudinal column (LC1) has a collective spacing of discrete bond units (CSP). In the laminate of the present invention, at least about 30%, preferably at least about 50%, of the longitudinal columns (LC) have a collective spacing of discrete bond units (CSP) that are not constant. While not required, all of the longitudinal columns (LC) may have a non-constant collective spacing of discrete bond units. The collective spacing of discrete bond units (CSP) of any adjacent longitudinal columns (LC1, LC2) are different from each other. For example, longitudinal column LC1 and longitudinal column LC2 do not have the same collective positioning of the discrete bond units (234). By the collection of discrete bond units (234) in a plurality of the longitudinal columns (LC), a 2-dimensional pattern of discrete bond units are provided.

Still referring to FIG. 3B, each of the plurality of longitudinal columns (LC) may comprise a first spacing (SP1) and a second spacing (SP2) wherein the second spacing (SP2) is greater than the first spacing (SP1), wherein a constant number of at least 2 discrete bond units (234) spaced apart with the first spacing (SP1) form a first array (AR1), wherein the first array (AR1) is spaced apart with each other with the second spacing (SP2). From about 2 to about 10 discrete bond units (234) may form the first array (AR1), and the second spacing (SP2) may be from about 3 mm to about 15 mm. When observing a plurality of longitudinal columns (LC1, LC2, LC3), the longitudinal position of the first array (AR1) may alternate in the transverse direction. Depending on the desired pattern of discrete bond units, the longitudinal columns (LC) may be spaced apart in the transverse direction at a pitch (TP) of from about 3 mm to about 15 mm. The pitch in the transverse direction (TP) of the longitudinal columns (LC) may be constant.

Referring to FIG. 3C, the discrete bond units (234) across the plurality of longitudinal columns (LC) may be so configured to provide an angled alignment (AA1, AA2), the angled alignment being a linear or a curved continuation of a plurality of discrete bond units (234) across the plurality of longitudinal columns (LC). As in FIG. 3C, the angled alignment (AA1, AA2) may be a linear continuation. The angled alignment (AA) may have an angle of from about 15 degrees to about 75 degrees, preferably from about 30 degrees to about 60 degrees, against the longitudinal axis. The angled alignment may be provided in a first angled alignment (AA1) and a second angled alignment (AA2), wherein the first angle (θ1) of the first angled alignment (AA1) and the second angle (θ2) of the second angled alignment (AA2) are in linear symmetry to the longitudinal axis, and wherein the first and second angled alignments (AA1, AA2) are repeated at a constant pitch in the longitudinal direction (LP) to provide a pattern of repeating rhomboid shapes or diamond shapes. The constant pitch in the longitudinal direction (LP) may be from about 6 mm to about 25 mm.

Referring to FIG. 3D, the discrete bond units (234) across the plurality of longitudinal columns (LC) may be so configured to comprise a first angled alignment (AA1) and a second angled alignment (AA2), wherein the first angle (θ1) of the first angled alignment (AA1) and the second angle (θ2) of the second angled alignment (AA2) are in linear symmetry to the longitudinal axis, and wherein the first and second angled alignments (AA1, AA2) are spaced apart with a longitudinal alignment (LA), wherein the collection of the plurality of discrete bond units formed by the first angled alignment (AA1), the longitudinal alignment (LA), and the second angled alignment (AA2) are repeated at a constant pitch in the longitudinal direction (LP) to provide a pattern of repeating hexagon shapes. The constant pitch in the longitudinal direction (LP) may be from about 5 mm to about 45 mm.

Referring to FIG. 3E, the discrete bond units (234) across the plurality of longitudinal columns (LC) may be so configured to provide a pattern of sinusoidal curve extending in the longitudinal direction.

Referring to FIG. 3F, the discrete bond units (234) across the plurality of longitudinal columns (LC) may be so configured to provide a plurality of angled alignments (AA1, AA2) provided in a pattern of repeated zigzag lines extending in the longitudinal direction.

The aforementioned patterns of discrete bond units depicted in FIGS. 3B-3F may be planned to have the following dimensions for providing the intended patterns.

| | FIG. | FIG. | FIG. | FIG. | FIG. |
|---|---|---|---|---|---|
| | 3B | 3C | 3D | 3E | 3F |
| Longitudinal dimension (mm) | 1 | 1 | 1 | 1 | 1 |
| Transverse dimension (mm) | 1 | 1 | 1 | 1 | 1 |
| Percentage of longitudinal columns | 100 | 76 | 60 | 67 | 100 |
| having non-constant spacing (%) | | | | | |
| First spacing SP1 (mm) | 1 | NA | 1 | 1 | 1 |
| Second spacing SP2 (mm) | 7 | NA | 12 | 21 | 10 |
| Pitch of transverse spacing TP (mm) | 10 | NA | NA | 23 | 5 |
| Angle of angled alignment (degrees) | NA | 45 | 30 | 45 | NA |
| Longitudinal pitch of shape LP (mm) | NA | 20 | 12 | 23 | 12 |

As a result of the above mentioned configuration of the discrete bond units as in FIGS. 3B-3F, the total bond area provided by the discrete bond units (234) may be controlled to be smaller than the configuration where the discrete bond units (234) are provided, such as in FIG. 3A, in a constant pitch in both the longitudinal and transverse directions. Without being bound by theory, it is believed that by having a smaller total bond area provided by the discrete bond units (234), and thus less adhesive applied between the inner sheet (94) and outer sheet (92), this contributes in creating larger gathers, and gathers of improved softness. Further, by controlling the shape of the gathers, the gathers may intuitively connote a certain function of the laminate.

Further, having a smaller total bond area provided by the discrete bond units (234) results in less restriction for the inner sheet (94) and the outer sheet (92), as well as the inner and outer sheets (92. 94) against the elastic members (96). Without being bound by theory, it is believed that by having less restriction for the inner and outer sheet materials (92, 94) against the elastic members (96), this allows improved stretchability of the elastic members (96), which may provide ease of application.

Compared to elastic belts made only by elastic bonding (230) wherein all of the elastic members (96) are continuously bonded, the elastic belt (40) of the present invention may have a lower Stretch Circumference Force, according to the measurements herein. What is meant by Stretch Circumference Force is the loading force at a certain stretch level, which is believed to simulate initial stretch experience felt by the wearer or caregiver when inserting hands and stretch opening the article. Further, despite such relatively low Stretch Circumference Force, the elastic belt (40) of the present invention may maintain a suitable Fit Circumference Force, according to the measurements herein. What is meant by Fit Circumference Force is the unloading force at a certain stretch level, which is believed to simulate the force felt by the wearer while wearing the article. Accordingly, the article of the present invention has a Stretch Circumference Force of no more than about 6.5N, and a Fit Circumference Force of at least about 2.5N according to the measurements herein, wherein the ratio of the value of Stretch Circumference Force/Fit Circumference Force is less than about 2.5, preferably less than about 2.3. A relatively low value for the ratio of Stretch Circumference Force/Fit Circumference Force means that there is less difference between the loading and unloading force felt by the wearer, thus providing the sensorial qualities similar to that of an undergarment. Without being bound by theory, it is also believed that, by having less restriction for the inner and outer sheet materials (92, 94) against the elastic members (96), this improves the breathability of the overall laminate, which may enhance skin health. Without being bound by theory, it is also believed that the plurality of discrete bond units (234) provides a configuration wherein a greater percentage of the inner and outer sheet materials (92, 94) are available for forming the outer surfaces of the laminate when the elastic belt (40) is contracted, while the elastic members (96) remain positioned inside the thickness of the laminate. As such, the laminate is provided with improved loft and thickness, thus imparting improved comfort and softness when worn.

The bonding strength of the elastic bonding (230) and the plurality of discrete bond units (234) may be the same or may be varied. The elastic bonding (230) and the plurality of discrete bond units (234) may be provided by the same hot melt adhesive. Exemplary hot melt adhesives suitable for the present invention include tradenames H4376 and H2401 available from Bostik.

The article of the present invention may have left and right elastic bondings (230), wherein in this length between the left and right elastic bondings (230), the elastic member may be attached or not attached by the plurality of discrete bond units (234). Without being bound by theory, it is believed that by providing the elastic bonding (230) length as small as possible, this provides the elastic belt to have favorable soft feeling. By providing the elastic bonding (230) length as small as possible, this prevents aging of the adhesive on the elastic member (96). Aging of the adhesive may result in changing the intended force profile expected by the elastic member.

The article of the present invention may have a Belt Elastic Bonding Percentage of less than about 25%, or less than about 20%, or less than about 15%, according to the measurements herein. Referring to FIG. 3A, among all the elastic members (96) disposed on the front elastic belt region (84), less than about 25% of those elastic members are bonded to the laminate in active elasticity by a complete elastic bonding (230, 230T) along the length of active elasticity of the individual elastic member. When all of the elastic members are completely bonded by elastic bonding (230, 230T), then the Belt Elastic Bonding Percentage is 100%. For those laminates having a Belt Elastic Bonding Percentage of less than about 100%, those elastic members that are not completely bonded by elastic bonding (230, 230T) may further be subject of inspection of the percentage by which the specific elastic member is bonded by elastic bonding. The article of the present invention may have an Individual Elastic Bonding Percentage of from about 10% to about 75%, or from about 20% to about 60%, according to the measurements herein. Namely, among the individual elastic members which are not 100% bonded by elastic bonding (230, 230T), the percentage of each individual elastic member attached to the laminate by elastic bonding is inspected, and averaged. The lower the Individual Elastic Bonding Percentage, the less adhesive is used for bonding the elastic member to the laminate. Without being bound by theory, by providing the Individual Elastic Bonding Percentage in a given range, this provides secure bonding of the elastic strands to the laminate to endure further processing during manufacture as well as handling during usage, while also providing the overall laminate soft, and provide aesthetically pleasing regularity and continuity of gathers.

Referring to FIG. 2, for the belt-type pant, the proximal edges (90) of the front and back belt (84, 86) may be provided with an end seal in order to keep the inner and outer sheets (92, 94) closed at the proximal edges (90) and thus prevent elastic members (96) from being accessible. Such unaccessibility of elastic members (96) may be particularly advantageous when the article is for a young wearer. Alternatively or additionally, the elastic member (96) which is positioned closest to the proximal edge (90) may be provided with an elastic bonding (230) along the transverse dimension of the elastic member (96) in portions of active elasticity.

Referring to FIG. 2, the elastic member (96) may be made by a plurality of elastic strands (96) running parallel to each other in the transverse direction, wherein the laminate has a region wherein the elastic strands (96) have a longitudinal pitch of from about 3 mm to about 18 mm, or from about 3 mm to about 12 mm, or from about 3 mm to about 7 mm.

The front and back elastic belts (84, 86) may be made by running the continuous inner and outer sheet materials as well as the continuous elastic strands along the transverse axis of the article, and bonding them via the elastic bondings (230) and plurality of discrete bond units (234). During manufacture, the continuous inner and outer sheet materials and continuous elastic strands may be transferred in the machine direction, wherein the machine direction of manufacture matches the transverse axis TX of the article. In such manufacturing process, the plurality of discrete bond units (234) are provided discretely aligned in the cross machine direction and intermittently spaced apart by a pitch in the machine direction of manufacture.

The tensile stress (N/m) of the entirety of the front and back elastic belts (84, 86), respectively, may be profiled in order to provide the functional benefits of the present invention, such as ease of stretch and application, while also maintaining certain force during wear, to prevent the article from sagging after loading. When the elasticity of the front and back elastic belts (84, 86) are provided by a plurality of elastic members (96) running in the transverse direction, the tensile stress may be adjusted by one or more of the following methods; 1) elongation rate of the elastic member (96); 2) density (dtex) of the elastic member (96); 3) longitudinal pitch of multiple elastic members (96); and 4) effective length of elasticity of the elastic member (96) in the transverse direction. By elongation, “0% elongation” is meant the original length of the elastic member. When a portion of an elastic member (96) is removed of its elasticity, the remainder of the intact elastic member capable of imparting elasticity is defined as the “effective length of elasticity of an elastic member”.

Referring to FIG. 2, the front and back elastic belts (26, 28) may each be divided into 4 zones spanning in the transverse direction and defined of its position from the distal edge (88) to the proximal edge (90) relative to the percentage of the seam length LS. In the example of FIG. 2, the entirety of the length of the belt side edge (89) of the front region (26) is the front belt (84), and is seamed with a certain length of the belt side edge (89) of the back region (28) which is the back belt (86) to define a seam length LS. When seam length LS is considered 0% at the distal edge (88) and 100% at the proximal edge (90) of the side seam (32), the zones are defined as such: 0-25% is the waist zone (102), 25-50% is the distal tummy zone (104), 50-85% is the proximal tummy zone (106), and 85-100% is the leg zone (108). When there is an elastic member disposed at 25% from the distal edge (88), such elastic member is considered to be included in the waist zone (102). When there is an elastic member disposed at 50% from the distal edge (88), or 85% from the distal edge (88), such elastic member is considered to be included in the proximal tummy zone (106).

Certain zones of the belt may be disposed of elastic bodies having a density of no more than about 500 dtex. Elastic bodies having a density of no more than about 500 dtex may be disposed on one or more of the front waist zone (102), the front distal tummy zone (104), or the front leg zone (108). Elastic bodies having a density of no more than about 500 dtex may be disposed on one or more of the back waist zone (102), the back proximal tummy zone (104), or the back leg zone (108). At least 50% of the elastic members on each of the front belt (84) and the back belt (86) may have a density of no more than about 500 dtex. Without being bound by theory, it is believed that elastic bodies of relatively low density impart an easy initial stretch experience when stretch opening the article (20), while maintaining a good fit during wear. Namely, use of such elastic bodies of relatively low density are advantageous in providing a controlled Stretch Circumference Force, while maintaining a certain Fit Circumference Force.

In the article of the present invention, the tensile stress of the front proximal tummy zone (106) may be provided higher than the tensile stress of any of the front waist zone (102), the front distal tummy zone (104), or the front leg zone (108). The tensile stress of the front proximal tummy zone (106) may be higher than the tensile stress of any other zone, either in the front or the back. The tensile stress of the back distal tummy zone (104) may be provided higher than any of the tensile stress of the back waist zone (102), the back proximal tummy zone (106), or the back leg zone (108). When comparing the 4 zones each of the front belt and the back belt, the tensile stress may be provided greatest in the order of: the front proximal tummy zone (106), followed by the back distal tummy zone (104). Without being bound by theory, such profiling of the tensile stress per zone is believed to provide the article of the present invention with a shaped elastic belt (40) that conforms well to a human body, particularly to a lower torso of a child of less than 36 months of age, and therefore provide good fit and comfort to the wearer, without compromise of sagging prevention or leakage prevention. Namely, the front proximal tummy zone (106) is subject to high tensile stress such that the article may be anchored against the wearer's trochanter, while leaving more area for the back proximal tummy zone (106) to accommodate the wearer's buttock. As long as the article is anchored securely at the trochanter, the waist zone (102) may be provided in relatively lower tensile stress, contributing in providing the waist zone (102) with soft fit.

For the belt-type pant, the longitudinal length LB of the back elastic belt (86) and the longitudinal length LF of the front elastic belt (84) may be provided the same, or the back elastic belt (86) may have a greater longitudinal length LB as in FIG. 2. Referring to FIGS. 1B and 2, when the wearable article is assembled to form the waist opening and the leg openings, the wearable article (20) is folded along the transverse centerline TX such that the front distal edge (88) is aligned with the back distal edge (88). The front side edge (89) is also aligned with a portion of the back side edge (89). Then the front belt (84) and the back belt (86) are joined at the front and back side edges (89) at the seams (32). The front and back proximal edges (90), however, may not be aligned to one another. The back proximal edge (90) may be disposed longitudinally closer than the front proximal edge (90) relative to the transverse center line TX such that the proximal portion of the back side panel (82) extends toward the crotch panel (56) of the main body (38) beyond the front proximal edge (90). The side edge of the proximal portion of the back side panel (82) may not be joined to anywhere and free from attachment. Thus, the proximal portion of the back side panel (82) provides a buttock cover (95), as in FIG. 1B.

The outer sheet (92) of the present invention may be a nonwoven having a basis weight of from about 10 gsm to about 55 gsm, or from about 10 gsm to about 35 gsm, and may have a fiber diameter of from about 0.8 dpf to about 6 dpf. The fiber diameter is described in denier per filament (dpf) used in the industry, which is grams/9,000 meters of length of fiber. The outer sheet (92) nonwoven may be made by processes such as spunbond, spunlace, carded or air-laid; and may comprise fibers and/or filaments made of polypropylene (PP), polyethylene (PE), polyethylene phthalate (PET), polylactic acid/polylactide (PLA) or conjugate fibers (such as PE/PET, PE/PP, PE/PLA) as well as natural fibers such as cotton or regenerated cellulosic fibers such as viscose or lyocell. The outer sheet (92) nonwoven may be a multilayer or composite structure combining nonwovens made by different processes and fibers such as combining spunbond and carded nonwovens. The outer sheet (92) nonwoven may be made by biodegradable material, or derived from renewable resources. Exemplary material for the outer sheet (92) include: air-through carded nonwoven having a thickness of at least about 50 μm, or at least about 80 μm, or at least about 200 μm. Such material may provide a soft lofty feeling to the garment-facing side. Suitable for the outer sheet (92) nonwoven of the present invention are air-through carded nonwoven material made of co-centric bicomponent fiber, crimping fiber made through core eccentric bicomponent filament or side by side bicomponent filament. Non-limiting examples of materials suitable for the outer sheet (92) nonwoven of the present invention include: 12-45 gsm air-through carded nonwoven substrate comprising PE/PET bi-component fibers, such as those available from Beijing Dayuan Nonwoven Fabric Co. Ltd. or Xiamen Yanjan New Material Co. Ltd., and 8-45 gsm spun melt nonwoven substrate comprising PP monofilament or PE/PP bi-component fibers, such as those available from Fibertex or Fitesa.

The inner sheet (94) of the present invention may be a nonwoven having a basis weight of from about 5 gsm to about 45 gsm, or from about 5 gsm to about 35 gsm. The inner sheet (94) nonwoven may have a fiber diameter of from about 0.5 dpf to about 4 dpf. The inner sheet (94) nonwoven may be made by processes such as spunbond, spunlace, carded or air-laid; and may comprise fibers and/or filaments made of polypropylene (PP), polyethylene (PE), polyethylene phthalate (PET), polylactic acid/polylactide (PLA) or conjugate fibers (such as PE/PET, PE/PP, PE/PLA) as well as natural fibers such as cotton or regenerated cellulosic fibers such as viscose or lyocell. The inner sheet (94) nonwoven may also be a multilayer or composite structure combining nonwovens made by different processes and fibers such as combining spunbond and carded nonwovens. The inner sheet (94) nonwoven may be made by biodegradable material, or derived from renewable resources. Non-limiting examples of materials suitable for the inner sheet (94) nonwoven of the present invention include: 12-30 gsm air-through carded nonwoven substrate made of PE/PET bi-component staple fiber, such as those available from Beijing Dayuan Nonwoven Fabric Co. Ltd. or Xiamen Yanjan New Material Co. Ltd., and 8-30 gsm spun melt nonwoven substrate comprising PP monofilament or PE/PP bi-component fibers, such as those available from Fibertex or Fitesa.

The basis weight of the outer sheet (92) and the inner sheet (94) may be adjusted such that the basis weight of the inner sheet (94) is not greater than the basis weight of the outer sheet (92). Thus, the outer sheet (92) may be provided with a soft lofty tactile sense which connotes high quality, while the inner sheet (94) may be kept thinner and conforming to the outer sheet (92), thus saving cost. Further, without being bound by theory, by providing the basis weight relationship as such, it is believed that skin sweating is effectively transported to the outer sheet (92) and outside the laminate, while preventing the transported sweat back to the inner sheet (94). The hydrophilicity/hydrophobicity of the outer sheet (92) and the inner sheet (94) may be adjusted such that the hydrophilicity of the outer sheet (92) is higher than that of the inner sheet (94). Without being bound by theory, it is believed that such gradient of hydrophilicity is advantageous in transporting skin sweat from the inner sheet (94) to the outer sheet (92) and outside the laminate. The inner sheet (94) nonwoven may be inherently hydrophobic. The inner sheet (94) nonwoven may be provided hydrophobicity by treating with hydrophobic melt additives into polymer resin in the fiber making process, or by applying hydrophobic additives after the nonwoven is formed. The outer sheet (92) nonwoven may inherently be hydrophobic, and thus provided relatively more hydrophilic than the inner sheet (94) by treating with hydrophilic melt additives into polymer resin in the fiber making process, or by applying hydrophilic additive after the nonwoven is formed.

Referring to FIG. 1B, the front elastic belt (84) may comprise a waist opening gather zone (220) along the waist opening for from about 5% to about 30%, or from about 10% to about 25%, of the longitudinal dimension of the front elastic belt LF, wherein the waist opening gather zone (220) is in active elasticity and provided with an artwork. The front elastic belt (84) may comprise a pair of leg opening gather zones along at least part of the leg openings for from about 5% to about 25%, or from about 5% to about 20%, of the longitudinal dimension of the front elastic belt LF, wherein the leg opening gather zones are in active elasticity and provided with an artwork. The artwork for the leg opening gather zones may be provided only along the leg opening, or spanned along the proximal edge (90) of the front elastic belt (84). The artwork for the leg opening gather zones may match the regions where the proximal edge of the front elastic belt (84) is in active elasticity, thus spanning slightly beyond the leg opening, while being discontinued in the regions matching the non-elastic region (221) of the front elastic belt (84). The longitudinal dimension of the leg opening gather zone may be from about 30% to about 80% of that of the waist opening gather zone (220).

The artwork of the waist opening gather zone (220) and the artwork of the leg opening gather zones of the front elastic belt (84) may have at least a common color or a common shape. For example, the artwork may be a line or a plurality of lines spanning in the transverse direction. The lines may be straight lines or wavy lines. By providing the waist opening gather zone (220) and leg opening gather zone of the front elastic belt (84) in similar appearance, the underwear like appearance of the overall article may be enhanced. The back elastic belt (86) may comprise a waist opening gather zone in active elasticity and provided with an artwork which matches the longitudinal dimension and appearance of those of the front elastic belt (84). The back elastic belt (86) may comprise a leg opening gather zone in active elasticity along the proximal edge (90) of the front elastic belt (84) and provided with an artwork which matches the longitudinal dimension and appearance of those of the front elastic belt (84). By providing both the front and back elastic belts (84, 86) with similar artwork, the underwear like appearance of the overall article may be enhanced.

### Belt Elastic Bonding Percentage and Individual Elastic Bonding Percentage

This measurement is for identifying 1) the percentage of elastic members on a laminate which are attached to the laminate solely by elastic bonding, defined as “Belt Elastic Bonding Percentage” and 2) the percentage of each individual elastic member attached to the laminate by elastic bonding, defined as “Individual Elastic Bonding Percentage”.

1. The test is performed in a room maintained at 23±2° C. and 50±5% relative humidity. The finished wearable article (20) sample is conditioned for more than 1 hour in the test room.
2. The front elastic belt region (84) is to be examined with the side seam kept intact and attached to the front belt. Cut the back belt in the longitudinal direction about 20-50 mm away from both side seams by scissors to open the belt from the back side. The opened belt is then carefully removed from the central chassis by hand so as not to alter the active elasticity region of the laminate. Therefore, it is acceptable to leave a layer from the central chassis attached to the laminate sample if the laminate cannot be separated from the central chassis. Cold spray is not used.
3. Observe the laminate sample thus obtained to identify a non-elastic region obtained by tummy cut. There may be samples which do not have a non-elastic region, and for such laminate sample the remainder of this step is skipped, and there exists no “T sample” hereinafter. The portion of the laminate sample extending in the lateral direction that has a non-elastic region is considered a tummy cut region (T) and the remainder of the laminate sample is considered a waist region (W). Separate the waist region (W) and tummy cut region (T) by cutting in the transverse direction by scissors at the middle point of the elastic members closest to each region.
4. For both the W (waist region) sample and the T (tummy cut region) sample thus obtained, the inner edge of the side seams are marked by a ballpoint pen. For the T sample, there exist 2 active elasticity regions. The starting point of the active elasticity region adjacent the non-elastic region is identified by observing the belt gathers caused by active elastic shrinkage, and marked by a ballpoint pen. The total number of elastic members on each sample is counted (“Total Elastic”). Those elastic members which are curved (for example, those extending in the longitudinal direction around the leg opening which do not have direction of stretch in the transverse direction) or which overlap with each other are not counted.
5. Each W and T sample is gently stretched until the gathers of the active elasticity region just disappear, then the length of the elastic length of each elastic member is measured to the nearest 1 mm by a scale. For the W sample, the distance between one side of seam inner edge to the other side of seam inner edge is measured for each elastic and summed up (“Total Elastic Width”). For the T sample, the distance between one side of seam inner edge to the marked active elasticity starting points are measured for both active elasticity regions, and summed up (“Total Side Width”).
6. For both W and T samples, cut the sample approximately along the longitudinal center of each active elasticity region to remove the stretching force. There is one active elasticity region on the W sample, and two active elasticity regions on the T samples, thus this step generates 2 cut W samples and 4 cut T samples.
7. Stretch the cut samples approximately up to the Full Stretch Circumference W1 according to the “Whole Article Force Measurement” for 5 times by hand, enough to make the elastic members slip away from the laminate, however, not beyond the point where the stretching damages the sheet materials constructing the samples. It should be noted that there is a possibility that there may be no slipping of elastic members.
8. Mark each individual elastic at the point up to where active elastic remains after the slip by observing belt gathering by active elastic shrinkage.
9. The cut samples are again gently stretched until the gathers just disappear, and then the remaining active elastic length of each individual elastic member is measured to the nearest 1 mm by a scale and summed up (“Total Elastic Bonding Width”).
10. The number of elastics members which keep initial active elastic length without slip is counted (“Full bonded Elastic”). For the cut W sample, a particular elastic member is considered as “slipped” if one of the left or right side cut sample of the elastic member slips. For the cut T sample, a particular elastic member is considered as “slipped” if any of the elastic portions of the cut samples is slipped.
11. “Individual Elastic Bonding Percentage” (%) is the length (mm) of “Total Elastic Bonding Width” divided by [length (mm) of “Total Elastic Width”+length (mm) of “Total Side Width”] reported to the nearest 1%.
12. “Belt Elastic Bonding Percentage” (%) is the number of “Full bonded Elastic” divided by the number of “Total Elastic” reported to the nearest 1%.

### Whole Article Force Measurement

Force is measured using an Electronic Tensile Tester with a computer interface such as the MTS Criterion C42 running TestWorks 4 Software (available from MTS SYSTEMS (CHINA) CO., LTD) or equivalent instrument. A load cell is selected so that force results for the samples tested will be between 10 and 90% of capacity of the load cell used. The instrument is calibrated according to the manufacturer's instructions. All testing is performed in a room maintained at 23±2° C. and 50±5% relative humidity.

The tensile tester is fitted with hanger-type sample holding fixtures (300) as shown in FIG. 6. Each fixture comprises a rigid linear rubber-coated horizontal bar section (302) to prevent sample slippage during testing. The outer bar diameter (including the rubber coating) of the horizontal bar sections is 10.0 mm. The central axes of the horizontal bar sections (302) are configured to remain parallel and in the same vertical plane throughout the test procedure. The gauge circumference is determined by the following equation:

Gauge Circumference=2×(H+D+πD/2)

where H is the vertical gap between the horizontal bar sections (302), and D is the outer diameter of the bar.

The instrument is set up to go through the following steps:

| Crosshead Speed | 254.0 | mm/min |
|---|---|---|
| Final Load Point | 19.61 | N |
| Hold Time | 0 | |
| Number of Cycles | 1 | |
| Data Acquisition Rate | 50 | Hz |

An article (20) sample is inserted onto the upper horizontal bar section (302) so that the bar passes through the waist opening and one leg opening of the article. The crosshead is raised until the specimen hangs above the lower bar and does not touch lower bar (302). The load cell is tared and the crosshead is lowered to enable the lower bar (302) to be inserted through the waist opening and other leg opening without stretching the article. The article is adjusted so that the longitudinal centerline LX of the article is in a horizontal plane halfway between the upper and lower bars (302). The center of the side portion in contact with the bar (302) is situated on the same vertical axis as the instrument load cell. The crosshead is raised slowly while the article is held in place by hand as necessary until the force is between 0.05 and 0.1N, while taking care not to add any unnecessary force. The gauge circumference at this point is the Initial Gauge Circumference. The test is initiated and the crosshead moves up at 254 mm/min until a force of 19.6 N is attained, then the crosshead immediately returns to the Initial Gauge Circumference at the same speed. The maximum circumference at 19.6 N and the force at 70% of the maximum circumference during the loading segment and unloading segment of the test are recorded.

The maximum circumference (mm) at 19.6 N is defined as the Full Stretch Circumference W1. The Full Stretch Circumference (mm)×0.7 is defined as the 70% Stretch Circumference W2. The force (N) during the loading segment of the test at 70% Stretch Circumference is defined as the Stretch Circumference Force. The force (N) during the unloading segment of the test at 70% Stretch Circumference is defined as the Fit Circumference Force. Five samples are analyzed and their average are calculated and reported to the nearest 1 mm or 0.01 N, respectively.

### Examples

Examples 1-2 and Comparative Example 1 are obtained as such. Examples 1-2 and Comparative Example 1 were subject to the tests described below.

Example 1: Size 4 (L-size) belt-type pant article having the configuration and elastic bonding of FIG. 3A, pattern of discrete bond units of FIG. 3B, and elastic profile and other properties of Tables 1 and 2 below.

Example 2: Size 4 (L-size) belt-type pant article having the configuration and elastic bonding of FIG. 3A, pattern of discrete bond units of FIG. 3C, and elastic profile and other properties of Tables 1 and 2 below.

Comparative Example 1: Size 4 (L-size) belt-type pant article having the configuration, elastic bonding, and pattern of discrete bond units of FIG. 3A, and elastic profile and other properties of Tables 1 and 2 below.

TABLE 1

| | Examples | Comparative |
|---|---|---|
| | 1 and 2 | Example 1 |
| Zone | Dtex/elongation %/number of elastic members | |
| Front waist zone | 470Dtex/180%/6 | 470Dtex/160%/4 |
| Front distal tummy | 470Dtex/180%/2 | 470Dtex/160%/2 |
| zone | with tummy cut (*1) | 470Dtex/230%/2 |
| | | with tummy cut (*1) |
| Front proximal tummy | 940Dtex/190%/8 | 940Dtex/230%/8 |
| zone | with tummy cut (*1) | with tummy cut (*1) |
| Front leg zone | 470Dtex/180%/2 | 470Dtex/130%/2 |
| | with tummy cut (*1) | with tummy cut (*1) |
| Back waist zone | 470Dtex/180%/4 | 470Dtex/160%/4 |
| Back distal tummy zone | 940Dtex/130%/4 | 940Dtex/160%/4 |
| Back proximal tummy | 470Dtex/180%/4 | 470Dtex/230%/4 |
| zone | | 470Dtex/270%/4 |
| | | with tummy cut (*1) |
| Back leg zone | 470Dtex/240%/4 | 470Dtex/270%/2 |
| | with tummy cut (*1) | with tummy cut (*1) |
| Outer sheet apertured | No | Yes |

(*1) “Tummy cut” in Table 1 refers to deactivation of elasticity at the transverse central area of elastic strands resulting in 68% effective length of elasticity.

The laminate for providing the front and back belt comprises discrete bond units, with the following patterns. The longitudinal dimension and transverse dimension of the discrete bond unit, and the percentage of longitudinal columns having non-constant spacing (NCS) were measured. Where applicable, the first spacing, the second spacing, and pitch of transverse spacing were measured. Where applicable, the angle of the angled alignment against the longitudinal axis, the pitch of the longitudinal spacing, and the longitudinal dimension of the given shape is measured.

TABLE 2

| | | | Comparative |
|---|---|---|---|
| | Example 1 | Example 2 | Example 1 |
| Longitudinal dimension (mm) | 1 | 1 | 1 |
| Transverse dimension (mm) | 1 | 1 | 1 |
| Percentage of longitudinal columns | 100 | 76 | 0 |
| having non-constant spacing (%) | | | |
| First spacing SP1 (mm) | 1 | NA | NA |
| Second spacing SP2 (mm) | 7 | NA | NA |
| Pitch of transverse spacing TP | 10 | NA | 7 |
| (mm) | | | |
| Angle of angled alignment | NA | 45 | NA |
| (degrees) | | | |
| Longitudinal pitch of shape LP | NA | 20 | NA |
| (mm) | | | |

### 1. Technical Measurements

The Belt Elastic Bonding Percentage (BEB) and Individual Elastic Bonding Percentage (IEB) were measured according to the measurement methods herein. The Stretch Circumference Force (SCF) and Fit Circumference Force (FCF) were measured according to the “Whole Article Force Measurement” method herein. Results of the aforementioned measurements are found in Table 3.

TABLE 3

| | | | Comparative |
|---|---|---|---|
| | Example 1 | Example 2 | Example 1 |
| BEB (%) | 0 | 0 | 11 |
| IEB (%) | 25 | 25 | 50 |
| SCF (N) | 6.3 | 6.3 | 6.03 |
| FCF (N) | 2.8 | 2.8 | 3.58 |
| Ratio SCF/FCF | 2.25 | 2.25 | 1.68 |

### 2. Consumer Acceptance Test

57 panelists who were caregivers of babies using Size 4 (L size) pant diapers and having a mixture of usage experience of major brands of similar price range used in the test were recruited. There were about equal number of caregivers of boy and girl babies in the age group of 25-36 years old. 5 finished product test samples were provided to the panelist to touch and feel with their hands.

Each respondent was asked to fill in a questionnaire individually after touching the test sample one by one. In the questionnaire, there were 3 values as found in Table 4, and each respondent was requested to sort and rate the test samples against those values using the ratings from 1 to 10, which were scored as such: ‘1=poor, 10=excellent’. The scores were averaged. Among 5 test samples tested, information for Examples 1, Comparative Example 1 are extracted below in Table 4.

TABLE 4

| | | Comparative |
|---|---|---|
| Values | Example 1 | Example 1 |
| Overall Softness | 7.5 (*2) | 7.1 |
| Belt softness | 8.3 | 7.9 |
| Belt good stretchability | 8.6 (*2) | 7.7 |

(*2) These scores were statistically significantly better against Comparative Example 1 at 90% confidence level.

According to the test results in Tables 3 and 4, Example 1 which meets the requirements of the present invention has statistically significantly higher “Overall softness” and “Belt good stretchability” compared to Comparative Example 1, and being better in “Belt softness” compared to Comparative Example 1, while having favorable Stretch Circumference Force and Fit Circumference Force values. Of notice is that Example 1 scores significantly higher in “Belt good stretchability” despite having similar favorable Stretch Circumference Force and Fit Circumference Force values compared to Comparative Example 1. It is believed that the gathers provided by the unique pattern of discrete bond units of Example 1 provides an improved function intuitive appearance, which affected the consumer's perception of stretchability.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm” Further, every numerical range given throughout this specification includes every narrower numerical range that falls within such broader numerical range.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A wearable article continuous in a longitudinal direction and a transverse direction comprising a front elastic belt region, a back elastic belt region, a crotch region, a waist opening, and a pair of leg openings; the crotch region extending longitudinally between the front elastic belt region and the back elastic belt region;
wherein each of the front and back elastic belt region comprises a laminate comprising an inner sheet, an outer sheet, and a plurality of elastic members running in the transverse direction, the longitudinal dimension between adjacent elastic members forming an elastic spacing;
wherein the laminate further comprising an elastic bonding which continuously bonds the elastic members for at least about 10 mm in the direction of stretch in a region adjacent the side edges of the front and back elastic belt regions, and a plurality of discrete bond units disposed between the elastic bondings in the transverse direction, each discrete bond unit applied to at least one of the inner sheet and the outer sheet and having a longitudinal dimension of from about 0.5 mm to about 2.0 mm, and a transverse dimension of from about 0.5 mm to about 2.0 mm, wherein between any two discrete bond units, the discrete bond units have a longitudinal spacing of at least about 0.2 mm with each other, and a transverse spacing of at least about 0.2 mm with each other, wherein the plurality of discrete bond units form a plurality of longitudinal columns, wherein at least about 30% of the longitudinal columns have a collective spacing of discrete bond units that are not constant, and the collective spacing of discrete bond units of any adjacent longitudinal columns are different from each other;
wherein there is at least one discrete bond unit disposed in each elastic spacing;
wherein the laminate is bonded by heat at the side edges to form side seams, the side seams existing no distant than about 20 mm from the side edges, and the remainder of the laminate is substantially free of heat bonds,
wherein the article has a Belt Elastic Bonding Percentage of less than about 25%, and an Individual Elastic Bonding Percentage of from about 10% to about 75%, according to the measurements herein.

2. The wearable article of claim 1, wherein the wearable article has a Stretch Circumference Force of no more than about 6.5N, and a Fit Circumference Force of at least about 2.5N according to the measurements herein, wherein the ratio of the value of Stretch Circumference Force/Fit Circumference Force is less than about 2.5.

3. The wearable article of claim 1, wherein the elastic bonding and the plurality of discrete bond units are provided by the same hot melt adhesive.

4. The wearable article of claim 1, wherein each of the plurality of longitudinal columns comprises a first spacing and a second spacing wherein the second spacing is greater than the first spacing, wherein a constant number of at least 2 discrete bond units are spaced apart with each other with the first spacing to form a first array, wherein the first array is spaced apart with each other with the second spacing.

5. The wearable article of claim 4, wherein across the plurality of longitudinal columns, the longitudinal position of the first array alternates in the transverse direction.

6. The wearable article of claim 4, wherein from about 2 to about 10 discrete bond units form the first array, and the second spacing is from about 3 mm to about 15 mm.

7. The wearable article of claim 4, wherein the longitudinal columns are spaced apart in the transverse direction at a pitch of from about 3 mm to about 15 mm.

8. The wearable article of claim 1, wherein the discrete bond units across the plurality of longitudinal columns are so configured to provide an angled alignment, the angled alignment being a linear or a curved continuation of a plurality of discrete bond units across the plurality of longitudinal columns.

9. The wearable article of claim 8, wherein the angled alignment is a linear continuation having an angle of from about 15 degrees to about 75 degrees against the longitudinal axis.

10. The wearable article of claim 9, comprising a first angled alignment and a second angled alignment, wherein the first angle of the first angled alignment and the second angle of the second angled alignment are in linear symmetry to the longitudinal axis, and wherein the first and second angled alignments are repeated at a constant pitch in the longitudinal direction to provide a pattern of repeating rhomboid shapes.

11. The wearable article of claim 10, wherein the constant pitch in the longitudinal direction is from about 6 mm to about 33 mm.

12. The wearable article of claim 8, comprising a first angled alignment and a second angled alignment, wherein the first angle of the first angled alignment and the second angle of the second angled alignment are in linear symmetry to the longitudinal axis, and wherein the first and second angled alignments are spaced apart with a longitudinal alignment, wherein the collection of the plurality of discrete bond units formed by the first angled alignment, the longitudinal alignment, and the second angled alignment are repeated at a constant pitch in the longitudinal direction to provide a pattern of repeating hexagon shapes.

13. The wearable article of claim 12, wherein the constant pitch in the longitudinal direction is from about 5 mm to about 45 mm.

14. The wearable article of claim 8, wherein the angled alignment is provided in a pattern of repeated zigzag lines extending in the longitudinal direction.

15. The wearable article of claim 8, wherein the angled alignment is provided in a pattern of sinusoidal curve extending in the longitudinal direction.
